(19) United States
(12) Patent Application Publication
(10) Pub. No.: US 2005/0080475 A1
(43) Pub. Date: Apr. 14, 2005

(54) Stent delivery devices and methods

(75) Inventors: Bernard Andreas, Redwood City, CA (US); Jeffry J. Grainger, Portola Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: XTENT, INC. A Delaware corporation, Redwood City, CA (US)

(21) Appl. No.: 10/686,507

(22) Filed: Oct. 14, 2003

(51) Int. Cl. A61F 2/06

(52) U.S. Cl. 623/1.11

(57) ABSTRACT

Stent delivery devices and methods include a shuttle for carrying multiple stent segments, to avoid direct contact between the stent segments and an expandable member. A sheath is disposed over the expandable member such that when it is withdrawn, a distal portion of the expandable member is exposed to expand against the shuttle, thus expanding the shuttle and expanding and deploying a selected number of stent segments. To deploy a second selected number of stent segments, the sheath may be withdrawn further to expose more of the expandable member. Thus, one or more custom length stents may be deployed with a reduced risk of damage to the expandable member and the stent segments and enhanced ease and accuracy of stent placement at a treatment location.

### BACKGROUND OF THE INVENTION

1. Field of the Invention

The present invention relates generally to medical devices and methods. More particularly, the invention relates to apparatus and methods for independently delivering a plurality of luminal prostheses within a body lumen.

Stenting has become an increasingly important treatment option for patients with coronary artery disease. Stenting involves the placement of a tubular prosthesis within a diseased coronary artery to expand the arterial lumen and maintain the patency of the artery. Early stent technology suffered from problems with restenosis, the tendency of the coronary artery to become re-occluded following stent placement. In recent years, however, improvements in stent design and the advent of drug-eluting stents have reduced restenosis rates dramatically. As a result, the number of stenting procedures being performed in the United States, Europe, and elsewhere has soared.

Stents are delivered to the coronary arteries using long, flexible vascular catheters, typically inserted through a femoral artery. For self-expanding stents, the stent is simply released from the delivery catheter, and it resiliently expands into engagement with the vessel wall. For balloon expandable stents, a balloon on the delivery catheter is expanded which expands and deforms the stent to the desired diameter, whereupon the balloon is deflated and removed.

Despite many recent advances in stent delivery technology, a number of shortcomings still exist. For example, current stent delivery catheters are not capable of customizing the length of the stent in situ to match the size of the lesion to be treated. While lesion size may be measured prior to stenting using angiography or fluoroscopy, such measurements may be inexact. If a stent is introduced that is found to be of inappropriate size, the delivery catheter and stent must be removed from the patient and replaced with a different device of correct size. Moreover, current stent delivery devices cannot treat multiple lesions with a single catheter. If multiple lesions are to be treated, a new catheter and stent must be introduced for each lesion to be treated.

Additionally, currently available stent delivery devices are not well-adapted for treating vascular lesions that are very long and/or in curved regions of a vessel. Current stents have a discrete length that is relatively short due to their stiffness. If such stents were made longer, to treat longer lesions, they would not conform well to the curvature of vessels or to the movement of vessels on the surface of the beating heart. On the other hand, any attempt to place multiple stents end-to-end in longer lesions is hampered by the inability to maintain appropriate inter-stent spacing and to prevent overlap of adjacent stents. Such shortcomings in the prior art are addressed by the inventions described in U.S. patent application Ser. No. 10/412,714 (Attorney Docket No. 21629-000330), entitled “Apparatus and Methods for Delivery of Multiple Distributed Stents,” filed on Apr. 10, 2003; and U.S. patent application Ser. No. 10/637,713 (Attorney Docket No. 21629-000340), entitled “Apparatus and Methods for Delivery of Multiple Distributed Stents,” filed on Aug. 8, 2003; both applications assigned to the assignee of the present invention, and both applications being hereby incorporated fully by reference.

Even with improvements such as those described in the above-reference patent applications, further improvements in stent delivery devices and methods are still being sought. For example, many balloon-expandable stents are currently delivered by devices in which the stents are in direct contact with the balloon or other expandable member. Often, such stents are pushed or otherwise advanced along the expandable member in its deflated state, and the expandable member is then inflated to deploy the stents. Such direct contact between the stents and the balloon, along with advancement of the stents along the balloon, may sometimes cause damage to the balloon and/or one or more stents or coatings thereon. A balloon or other expandable member may also interfere with stent advancement, especially after the balloon has been inflated and deflated multiple times and, thus, becomes somewhat flaccid and/or deformed. Thus, stent delivery devices in which the stents directly contact the expandable member may lead to increased risk of balloon or stent damage, increased general wear and tear, difficult stent advancement along the delivery device, and less precise stent placement.

Therefore, a need exists for improved stent delivery devices and methods. Ideally, such devices and methods would reduce or eliminate direct contact between stents and the expandable member of the delivery device to reduce damage to the stents and expandable member and to facilitate stent placement. At least some of these objectives will be met by the present invention.

2. Description of the Background Art

U.S. patent application Ser. Nos. 10/412,714 and 10/637,713, previously incorporated by reference, describe apparatus and methods for delivery of multiple distributed stents. U.S. Pat. Nos. 6,485,510 and 6,258,117 to Camrud et al. describe segmented stents with breakable connections between the segments. U.S. Patent Application Publication No. 2002/0156496 (inventor Chermoni) describes a catheter for carrying stents including a stent positioner. U.S. Pat. No. 6,143,016 to Beam et al. describes a stent delivery sheath. U.S. Pat. No. 5,807,398 to Shaknovich describes a shuttle stent delivery catheter. U.S. Pat. No. 5,571,086 (Kaplan et al.) and U.S. Pat. No. 5,776,141 (Klein et al.) describe an expandable sleeve for placement over a balloon catheter for the delivery of one or two stent structures to the vasculature. U.S. Pat. No. 5,697,948 to Marin et al. describes a catheter for delivering stents covered by a sheath. Patent application serial numbers 2003/0139797 (Johnson) and 2003/0114919 (McQuiston) describe covered segmented stents.

### BRIEF SUMMARY OF THE INVENTION

Devices and methods of the present invention provide for delivering prostheses such as stents and grafts into body lumens. Generally, devices of the invention include a catheter having a stent shuttle for carrying stents in such a way that they do not directly contact an expandable balloon of the catheter. Stents (or stent segments) are typically deployed from the catheter by retracting a sheath and/or advancing the stents along the shuttle using a stent pushing device. An expandable balloon is then inflated to expand the shuttle, which in turn expands one or more stent segments. Such devices and methods may be used to individually deploy single stents, groups of stents, or single or multiple stent segments in a body lumen while avoiding direct contact between the expandable deployment balloon and the stents.

The terms “stents” and “stent segments” are used frequently in this application. The term “stent” is well known in the art, and some stents are segmented into two or more stent segments. Generally, adjacent stent segments of one stent may be connected, partially connected, breakably connected, or completely separate. Methods and apparatus of the present invention are generally used to deliver multiple stents, multiple stent segments or both in a body lumen such as a blood vessel. In various embodiments, for example, multiple stents each having multiple segments, multiple segments of one stent, and/or multiple non-segmented stents may be delivered. Oftentimes, the same embodiment of a device or method may be used to deliver multiple stents, multiple segments of one stent, multiple segments of multiple stents and/or the like. Therefore, the terms “stents” and “stent segments” may sometimes be used interchangeably throughout the application and such terms should not be interpreted to limit the scope of the invention in any way.

In one aspect of the invention, a stent delivery device for delivering a plurality of stent segments to a treatment site includes: a catheter shaft having a proximal end and a distal end; an expandable member coupled with the catheter shaft near the distal end; an axially movable sheath disposed directly or indirectly over at least part of the catheter shaft and the expandable member; a shuttle disposed coaxially over at least part of the catheter shaft and the expandable member, at least part of the shuttle being radially expandable; and a plurality of stent segments disposed along the shuttle. Generally, moving the sheath from an initial position where it covers some or all of the expandable member axially toward the proximal end of the catheter shaft exposes at least part of the expandable member, allowing it to expand against the shuttle to cause the shuttle to radially expand, thus causing at least one of the plurality of stent segments to expand. Exposing a selected number of stent segments enables deployment of a stent of custom length. In various embodiments, the sheath may be disposed over the shuttle, while in alternative embodiments the shuttle may be disposed over the sheath.

In one embodiment, the shuttle is disposed over the sheath, and the sheath is disposed over the expandable member. In an alternative embodiment, the sheath may be disposed over the shuttle, with the shuttle being disposed over the expandable member. In either case, moving the sheath axially toward the proximal end of the catheter shaft will expose (or further expose) at least part of the expandable member, allowing it to expand against the shuttle to cause the shuttle to radially expand, thus causing at least one of the plurality of stent segments to expand. In some embodiments, the at least one stent segment comprises a first selected number of stent segments. In some embodiments, a second selected number of stent segments may be deployed in subsequent steps. Thus, the device may be used not only to deploy one custom length stent but multiple custom length stents in some embodiments.

In some embodiments, the shuttle may be slidably disposed over the sheath and the catheter shaft. The stent segments may either be fixed to the shuttle until they are expanded into a deployed position or slidably disposed along the shuttle. In the latter case, the device may also include a stent pushing member disposed over the shuttle, proximal to the plurality of stent segments, for advancing the stent segments along the shuttle in a direction from proximal to distal. In such embodiments, an abutment may be provided at or near the distal end of the shuttle for preventing the plurality of stent segments from being advanced beyond the distal end of the shuttle.

In some embodiments where the sheath is disposed over the shuttle, the shuttle may be axially movable relative to the catheter shaft, so that the expandable member may be retracted relative to the shuttle to allow for multiple stent segment deployments. In other embodiments, the shuttle may be fixed in its position relative to the catheter shaft. Also in embodiments where the sheath is disposed over the shuttle, the sheath may further include at least one valve member at or near its distal end (or elsewhere along the sheath) for selectively retaining at least one stent within the sheath. Such a valve member is typically disposed on the sheath and is used for controlling the deployment of one or more stent segments, stent segments or other prostheses. The valve member may be distinguished from the abutment on the shuttle described above, in that the abutment is typically located on the shuttle and serves to prevent one or more stent segments, stent segments or other prostheses from being pushed off the distal end of the shuttle, such as with a pusher device.

In another aspect of the invention, a stent delivery device for delivering a plurality of stent segments to a treatment site comprises: a catheter shaft having a proximal end and a distal end; an expandable member coupled with the catheter shaft near the distal end; an axially movable sheath disposed over at least part of the catheter shaft and the expandable member; a shuttle disposed over at least part of the catheter shaft and the expandable member, at least part of the shuttle being radially expandable; a plurality of stent segments slidably disposed along the shuttle; and a stent pushing member disposed over the shuttle, proximal to the plurality of stent segments, for advancing the stent segments distally along the shuttle. Again, moving the sheath axially toward the proximal end of the catheter shaft will expose at least part of the expandable member, allowing it to expand against the shuttle to cause the shuttle to radially expand, causing at least one of the plurality of stent segments to expand.

In yet another aspect of the invention, a method for delivering a plurality of stent segments to a treatment site involves positioning a distal portion of a stent delivery catheter device at the treatment site and moving a sheath of the catheter device proximally relative to an expandable member on the catheter device, thus allowing at least part of the expandable member to expand against an expandable shuttle of the catheter device to deploy at least one of the plurality of stent segments. In some embodiments, the at least one stent segment comprises a first plurality of stent segments. Optionally, the method may further include moving the sheath farther proximally to further expose the expandable member to allow it to expand against the expandable shuttle to deploy at least a second plurality of stent segments. The method may then involve serially moving the sheath farther proximally to deploy third, fourth, fifth pluralities or any number of subsequent stent segments.

Generally, the method may be used to select any number of stent segments to deploy and to deploy that selected number of stent segments. In some embodiments, a second number of stent segments may then be selected and deployed, and a third number and so on. Thus, methods of the present invention provide for selection and deployment of custom length stents. Deployed stents may have different lengths, shapes, coatings, stiffness, strut configurations, geometries or the like.

Further aspects of the nature and advantages of the invention will become apparent from the detailed description below, in conjunction with the drawings.

### BRIEF DESCRIPTION OF THE DRAWINGS

FIG. 1 is a perspective view of a stent delivery catheter according to one embodiment of the present invention with a distal portion shown in cross section.

FIG. 2 is a side cross-sectional view of a distal portion of a stent delivery catheter having a shuttle with fixed prostheses and a sheath within the shuttle, according to one embodiment of the present invention.

FIG. 3 is a side cross-sectional view of a distal portion of a stent delivery catheter having a shuttle with fixed prostheses and a sheath outside the shuttle, according to one embodiment of the present invention.

FIG. 4 is a side cross-sectional view of a distal portion of a stent delivery catheter having a shuttle with slidable prostheses and a sheath within the shuttle, according to one embodiment of the invention.

FIG. 5 is a side cross-sectional view of a distal portion of a stent delivery catheter having a shuttle with slidable prostheses and a sheath outside the shuttle, according to one embodiment of the invention.

FIGS. 6A-6D demonstrate a method for delivering a plurality of prostheses at a treatment site, according to one embodiment of the invention.

### DETAILED DESCRIPTION OF THE INVENTION

Stent delivery devices of the present invention generally include a shuttle for carrying multiple stent segments, so that the stent segments need not be placed on, or advanced directly over, an expandable balloon member of the device. The shuttle is disposed over, and at least partly expandable by, an expandable member such as a balloon, to expand and deploy the stent segments. In some embodiments, the shuttle is positioned outside the expandable member and a sheath, such that when the sheath is withdrawn part of the expandable member is exposed to expand against the shuttle, thus expanding and deploying one or more stent segments. In other embodiments, the shuttle may be positioned inside the sheath such that withdrawing the sheath allows the expandable member and the shuttle to expand to deploy one or more stent segments. In various embodiments, stent segments may either be fixed on the shuttle or may be slidably disposed along the shuttle. In slidable embodiments, the device may further include a stent pushing member for advancing the stent segments distally along the shuttle and/or an abutment for preventing stent segments from being pushed off the distal end of the shuttle.

Shuttles of the present invention generally enhance stent delivery by providing for delivery of custom length stents while avoiding direct contact between the stent segments and the expandable balloon, thus reducing the risk of damage to the balloon and/or the stent segments and enhancing ease and accuracy of stent placement at a treatment location. By delivery of custom length stents, it is meant that a number of stent segments may be selected and deployed. For example, in some embodiments, multiple stents each having multiple stent segments may be disposed along a shuttle. A user may choose to deploy a selected number of stent segments of a first stent at a first location in a body lumen by withdrawing a sheath to expose the selected number of segments. The user may then choose to deploy a selected second number of stent segments, either from the first stent or from a subsequent stent, at a second location, and so on. In this way, stents of custom length, and possibly of other custom characteristics such as configuration, may be selected and deployed.

The terms “stents” and “stent segments” are used frequently in this application. The term “stent” is well known in the art, and some stents are segmented into two or more stent segments. Generally, adjacent stent segments of one stent may be connected, partially connected, breakably connected, or completely separate. Methods and apparatus of the present invention are generally used to deliver multiple stents, multiple stent segments or both in a body lumen such as a blood vessel. In various embodiments, for example, multiple stents each having multiple segments, multiple segments of one stent, and/or multiple non-segmented stents may be delivered. Oftentimes, the same embodiment of a device or method may be used to deliver multiple stents, multiple segments of one stent, multiple segments of multiple stents and/or the like. Therefore, the terms “stents” and “stent segments” may sometimes be used interchangeably throughout the application and such terms should not be interpreted to limit the scope of the invention in any way.

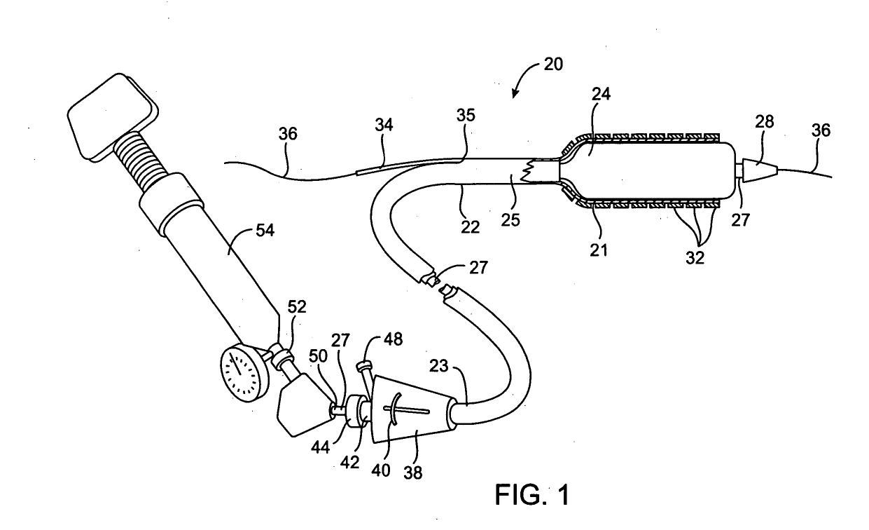

Referring now to FIG. 1, a stent delivery catheter device 20 is shown, with a distal portion in cross-section. In one embodiment, the catheter device 20 may be similar to a stent deliver catheter described in U.S. patent application Ser. No. 10/637,713, previously incorporated by reference, although it includes the added feature of a shuttle 21 along which multiple stent segments 32 are disposed. Again, one stent having multiple segments 32, multiple stents each having multiple segments 32, multiple unsegmented stents or the like may be disposed on shuttle 21. Generally, stent delivery catheter 20 may suitably include a catheter body 22 comprising a sheath 25 slidably disposed over a shaft 27. An expandable member 24, preferably an inflatable balloon (shown in an inflated configuration), is mounted to shaft 27 and is exposed by retracting sheath 25 relative to shaft 27. Alternatively, the expandable member could be any one of a variety of other mechanically, hydraulically, electrically, or otherwise expandable structures known in the intraluminal catheter arts, such as expandable braids, expandable cages, expandable Mallecott structures, self-expanding structures (including shape memory cages), and the like. A tapered nosecone 28, composed of a soft elastomeric material to reduce trauma to the vessel during advancement of the device, may be mounted distally of expandable member 24. Stent segments 32 are disposed on shuttle 21, which in turn is disposed on expandable member 24 for expansion therewith, typically being coaxially and slidably received over shaft 27. In some embodiments, a guidewire tube 34 is slidably positioned through a guidewire tube exit port 35 in sheath 25 proximal to expandable member 24. A guidewire 36 is positioned slidably through guidewire tube 34, expandable member 24, and nosecone 28 and extends distally thereof. Other designs where a guidewire is received through the entire shaft 27 are also within the present invention.

A handle or hub 38 is mounted to a proximal end 23 of sheath 25 and includes an actuator 40 slidably mounted thereto for purposes described below. An adaptor 42 is mounted to the proximal end of handle 38 and provides a catheter port 44 through which shaft 27 is slidably positioned. A flush port 48 is mounted to the side of adaptor 42 through which a fluid such as saline can be introduced into the interior of catheter body 22. An annular seal (not shown) in catheter port 44 seals around shaft 27 to prevent fluid from leaking through catheter port 44. Optionally, a clamp (not shown) such as a threaded collar, can be mounted to catheter port 44 to lock shaft 27 relative to handle 38. While adaptor 42 is shown separately from handle 38, the structures could be made integral to each other as well.

Shaft 27 has a proximal end 50 to which is mounted an inflation adaptor 52 (which could also be formed integrally with handle 38). Inflation adaptor 52 is configured to be fluidly coupled to an inflation device 54, which may be any commercially available balloon inflation device such as those sold under the trade name “Indeflator™,” available from Advanced Cardiovascular Systems of Santa Clara, Calif. Inflation adaptor 52 is in fluid communication with expandable member 24 via an inflation lumen in shaft 27 to enable inflation of expandable member 24. For further description of devices and methods for delivering distributed stents, as well as various embodiments of stents themselves, reference may be made to U.S. patent application Ser. Nos. 10/412,714 and 10/637,713, previously incorporated by reference.

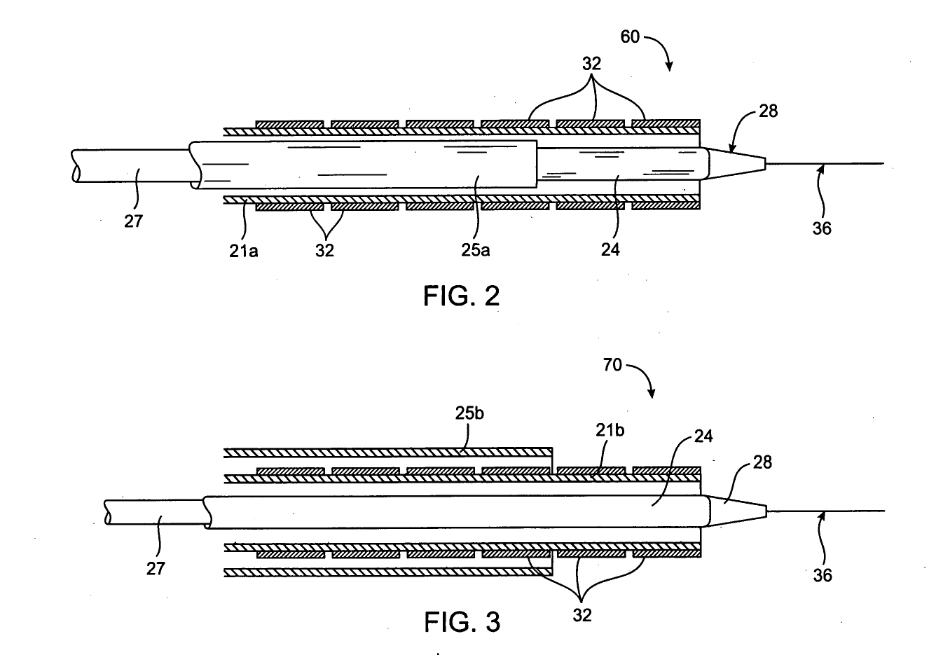

As mentioned above and described in more detail below, the configuration of stent delivery catheter 20 make take any of a number of alternative forms. For example, in FIG. 2 shuttle 21 is disposed within sheath 25a and around expandable member 24. In an alternative embodiment, shuttle 21 may be disposed outside of sheath 25b. In either of these embodiments, shuttle 21 may comprise a relatively long tubular member, perhaps extending much of the length of catheter 20, or alternatively may be a tubular member disposed along only a distal portion of catheter 20. Various shuttles 21 may be either fixed or slidable relative to shaft 27 and/or sheath 25. Stents 30 or stent segments 32 may be mounted on shuttle 21 in fixed or slidable fashion, in various embodiments, with slidable embodiments often including a stent pushing member for advancing the segments 32. Therefore, FIG. 1 depicts only one exemplary embodiment of a stent delivery device and in no way should be interpreted to limit the scope of the invention.

Stent segments 32 are described more fully in U.S. patent application Ser. No. 10/637,713, previously incorporated by reference and Application Ser. No. 60/440,839, filed Jan. 17, 2003 (Attorney Docket No. 21629-000500), which is incorporated herein by reference. In one embodiment, for example, each stent segment is about 2-8 mm in length, and up to 10-50 stent segments may be positioned end-to-end in a line over shuttle 21. Stent segments 32 may be in direct contact with each other, but preferably stent segments 32 are spaced apart from each other enough so that each stent segment 32 may be expanded without interfering with any adjacent stent segment(s) 32. Alternatively, separate spacing elements may be disposed between adjacent stent segments 32. Such spacing elements may be plastically deformable or self-expanding so as to be deployable with stent segments 32 into the vessel, but alternatively could be configured to remain on shuttle 21 following stent deployment; for example, such spacing elements could comprise elastic rings which elastically expand with balloon member 70 and resiliently return to their unexpanded shape when shuttle 21 is deflated.

Stent segments 32 are preferably a malleable metal so as to be plastically deformable by expandable member 24 as they are expanded to the desired diameter in the vessel. Alternatively, stent segments 32 may be formed of an elastic or super elastic shape memory material such as Nitinol so as to self-expand upon release into the vessel by retraction of sheath 25. Stent segments 32 may also be composed of polymers or other suitable biocompatible materials. In self-expanding embodiments, expandable member 24 may also be used for predilatation of a lesion prior to stent deployment or for augmenting the expansion of the self-expanding stent segments. In preferred embodiments, stent segments 32 are coated with a drug that inhibits restenosis, such as Rapamycin, Everolimus, Paclitaxel, analogs, derivatives, prodrugs, or derivatives of Rapamycin, Everolimus or Paclitaxel, or other suitable agent, preferably carried in a bioerodable polymeric carrier. Alternatively, stent segments 32 may be coated with other types of drugs and therapeutic materials such as antibiotics, thrombolytics, anti-thrombotics, anti-inflammatories, cytotoxic agents, anti-proliferative agents, vasodilators, gene therapy agents, radioactive agents, immunosuppressants, chemotherapeutics and stem cells. Such materials may be coated over all or a portion of the surface of stent segments 32, or stent segments 32 may include apertures, holes, channels, or other features in which such materials may be deposited.

Stent segments 32 may have a variety of configurations, including those described in Application Ser. No. 60/440,839, previously incorporated by reference. Stent segments 32 are preferably completely separate from one another without any interconnections, but alternatively may have couplings between two or more adjacent segments which permit flexion between the segments. As a further alternative, one or more adjacent stent segments 32 may be connected by separable or frangible couplings that are separated prior to or upon deployment, as described in U.S. application Ser. No. 10/306,813, filed Nov. 27, 2002 (Attorney Docket No. 21629-000320), which is incorporated herein by reference.

Referring now to FIG. 2, a distal portion of one embodiment of a stent delivery catheter 60 is shown. Again, delivery catheter 60 may suitably include catheter shaft 27, expandable member 24, sheath 25a and nosecone 28, and may allow for passage of a guidewire 36. Stent segments 32 are disposed along a shuttle 21a, and in this embodiment shuttle 21a is disposed over sheath 25a and expandable member 24.

Shuttle 21a may be composed of any suitable material or combination of materials and may have any suitable length, inner diameter, thickness and the like. Generally, at least part of shuttle 21a will be expandable so that expandable member 24 can expand shuttle 21a to expand and deploy stent segments 32. Shuttle 21 may thus be expandable along its entire length or only along a portion of its length near the distal end. The expandable portion of shuttle 21a may be composed of similar materials to that of the expandable member 24 or alternative materials. In some embodiments, for example, at least part of shuttle 21a may comprise a semi-compliant polymer such as Pebax or Nylon and is configured to resiliently return to its unexpanded shape following expansion. A non-expandable proximal section of shuttle 21a, if one is included, may be made of a polymer such as polyimide, PTFE, FEP or Pebax, or may comprise any other suitable material. To enhance axial sliding of sheath 25a, shuttle 21a may be made of a friction-reducing or friction-minimizing material and/or may be covered with a friction reducing coating.

Sheath 25a has a distal portion configured to surround expandable member 24 when in an unexpanded configuration. The distal portion may extend proximally to a junction, preferably aligned with the location of guidewire tube exit port, where the distal portion is joined to a proximal portion that extends proximally to handle 38 (see FIG. 1). In one embodiment, the distal portion has a length of about 15-35 cm, and the proximal portion has a length of about 100-125 cm. The proximal portion may be constructed of a variety of biocompatible polymers or metals, preferably being stainless steel or Nitinol. The distal portion may be a polymer such as PTFE, FEP, polyimide, or Pebax, and is preferably reinforced with a metallic or polymeric braid to resist radial expansion when expandable member 24 is expanded.

Preferably, the proximal portion has a smaller transverse dimension than the distal portion to accommodate the added width of a guidewire tube within the vessel lumen, as well as to maximize flexibility and minimize profile. In one embodiment, for example, the distal portion may have an outer diameter of about 1.0-1.5 mm, and the proximal portion may have an outer diameter of about 0.7-1.0 mm. At the junction of the proximal portion with the distal portion, a proximally-facing crescent-shaped opening may be formed between the two tubular members that creates a guidewire tube exit port. Excess space within the crescent-shaped opening may be filled with a filler material such as adhesive.

In some embodiments, shuttle 21 is slidably coupled with catheter 60 to allow it to move axially relative to one or more catheter components. Sheath 25a is withdrawn proximally to expose a portion of expandable member 24. Expandable member 24 (shown in unexpanded configuration) then expands to contact and expand shuttle 21a which in turn expands and deploys a selected number of stent segments 32. In this way, stent segments 32 may be expanded and deployed one at a time or in groups to provide custom length stent deployment. As sheath 25a is withdrawn farther proximally, more expandable member 24 is exposed, more shuttle 21a is expanded, and additional stent segments 32 are expanded and deployed. Catheter 60 may also be retracted relative to shuttle 21 to align expandable member 24 with additional stent segments 32. In other embodiments, shuttle 21a may be fixed to delivery catheter 60 so that it does not slide axially relative to catheter shaft 27, expandable member 24 and the like.

Referring now to FIG. 3, a distal portion of an alternative embodiment of a stent delivery catheter 70 is shown. In this embodiment, shuttle 21b is disposed within a sheath 25b, stent segments 32 are disposed along shuttle 21b, and expandable member 24 is disposed within shuttle 21b. In such an embodiment, sheath 25b may be retracted proximally to allow expandable member 24, shuttle 21b and stent segments 32 to expand. Alternatively, shuttle 21b and expandable member 24 may be advanced distally out of sheath 24. As with the previously described embodiment, as expandable member 24, shuttle 21b and stent segments 32 are exposed from sheath 25b, they may be expanded to deploy stent segments 32 within a vascular or other lumen.

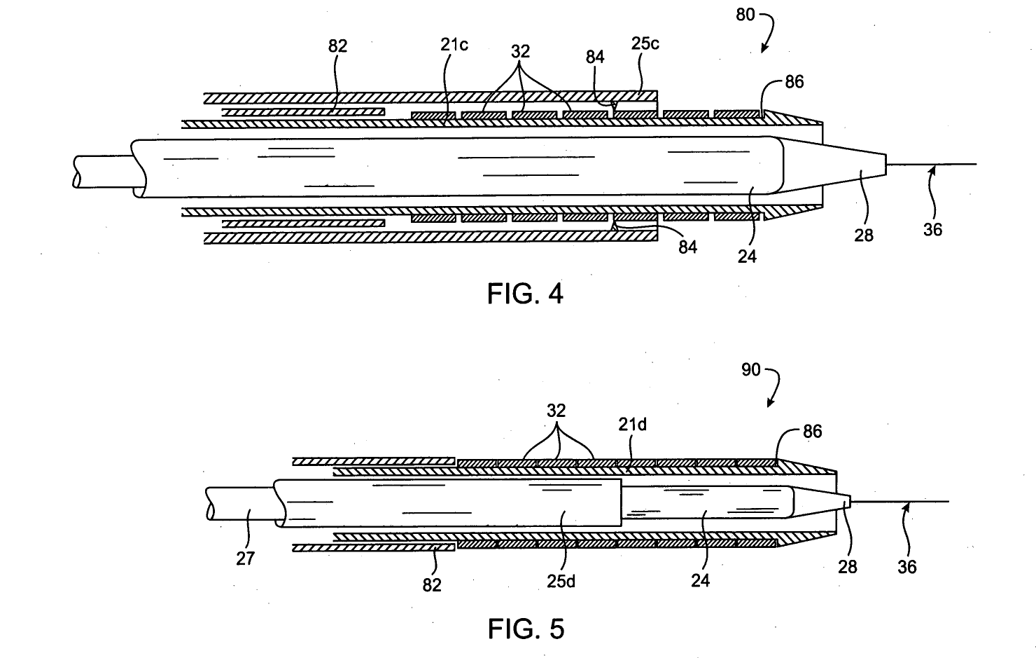

Turning now to FIG. 4, a distal end of another embodiment of a stent delivery catheter 80 has shuttle 21c again positioned within a sheath 25c. In this embodiment, however, stent segments 32 are slidably disposed along shuttle 21c. In such embodiments, stent segments 32 may be advanced along shuttle 21c using a proximally positioned stent pushing member 82. Stent pushing member 82 may be constructed of a variety of biocompatible polymers or metals, preferably being stainless steel or Nitinol. To prevent stent segments 32 from advancing too far and falling of the distal end of shuttle, an annular ridge 86 or other abutment may be included on shuttle 21c to act as a stop to the most distal stent segment 32. Such embodiments may also include one or more valves 84 disposed on the inner surface of sheath 25c for allowing a physician to better regulate the number of stent segments 32 that pass through sheath 25c. Such valves are described in copending U.S. patent application Ser. No. 10/412,714, which was previously incorporated by reference. Valve 84 also enables the physician to retract stent segments 32 within sheath 25c, thereby creating suitable spacing between segments 32 for deployment without interference between adjacent segments 32.

In another embodiment, with reference now to FIG. 5, a stent delivery catheter 90 includes axially slidable sent segments 32 on a shuttle 21d disposed outside of a sheath 25d. Again, a stent pushing member 82 is included in catheter device 90, and shuttle 21d includes a annular ridge 86. Sheath 25d is axially slidable over expandable member 24 to selectively expose a desired length of expandable member 24.

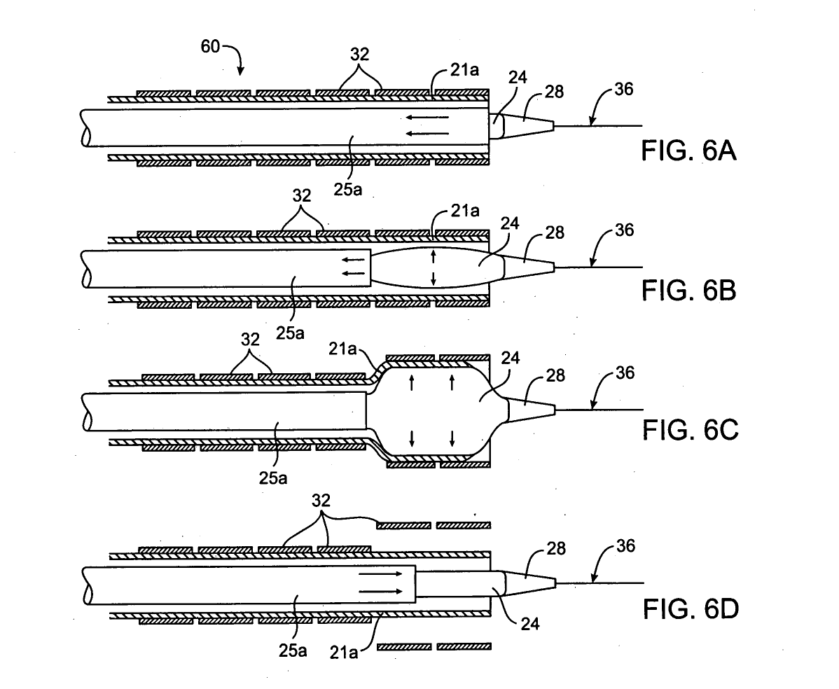

Referring now to FIGS. 6A-6D, a method for delivering stent segments is shown, though for purposes of clarity no vasculature or other lumen is shown. Generally, a stent delivery catheter 60 will be advanced through a patient's vasculature or other lumen to a desired location for delivering stent segments 32. At that point, sheath 25a may be withdrawn or retracted proximally, as shown by the two proximally directed arrows in FIG. 6A, to expose at least part of expandable member 24 within shuttle 21A. Exposed expandable member 24 may then be expanded, as shown in FIGS. 6B and 6C. Upon such expansion, expandable member 24 contacts and expands an expandable portion of shuttle 21a, which in turn causes one or more stent segments 32 to expand, as shown in FIG. 6C. When expandable member 24 is subsequently deflated, stent segments 32 remain expanded and in place, as shown in FIG. 6D. Shuttle 21a, however, resumes its original shape. A physician may then reposition delivery catheter 60 and retract sheath 25a and expandable member 24 further proximally and expand expandable member 24 and shuttle 21a to deploy additional stent segments 32. When a procedure is finished, a physician may advanced sheath 25a distally to cover expandable member 24. The method may further include advancing stent segments 32 with a stent pushing member, sliding shuttle 21a, and using a valve to control stent advancement, using the catheter embodiment of FIG. 4. Various embodiments of the method may be used by adding, subtracting or substituting steps without departing from the scope of the invention.

Although the above is complete description of the preferred embodiments of the invention, various alternatives, additions, modifications and improvements may be made without departing from the scope thereof, which is defined by the claims.

1. A stent delivery device for delivering a plurality of stent segments to a treatment site, the device comprising:
a catheter shaft having a proximal end and a distal end;
an expandable member coupled with the catheter shaft near the distal end;
an axially movable sheath disposed over at least part of the catheter shaft and the expandable member;
a shuttle disposed coaxially over at least part of the catheter shaft and the expandable member, at least part of the shuttle being radially expandable; and
a plurality of stent segments disposed along the shuttle;
wherein moving the sheath axially toward the proximal end of the catheter shaft allows at least part of the expandable member to expand against the shuttle to cause the shuttle to radially expand, thus causing at least one of the plurality of stent segments to expand.

2. A device as in claim 1, wherein the shuttle is slidably disposed over at least part of the catheter shaft and the expandable member.

3. A device as in claim 1, wherein the shuttle is fixedly disposed over at least part of the catheter shaft and the expandable member.

4. A device as in claim 1, wherein the shuttle is disposed over the sheath.

5. A device as in claim 1, wherein the sheath is disposed over the shuttle.

6. A device as in claim 1, wherein the sheath is adapted to expose a first portion of the expandable member to deploy a first selected number of stent segments.

7. A device as in claim 6, wherein the sheath is adapted to further expose at least a second portion of the expandable member to deploy a second selected number of stent segments.

8. A device as in claim 1, wherein the stent segments are fixed to the shuttle until they are expanded into a deployed position.

9. A device as in claim 1, wherein the stent segments are slidably disposed along the shuttle, the device further comprising a stent pushing member disposed over the shuttle, proximal to the plurality of stent segments, for advancing the stent segments along the shuttle in a direction from proximal to distal.

10. A device as in claim 9, wherein the shuttle further comprises an abutment at or near a distal end of the shuttle for preventing the plurality of stent segments from being advanced beyond the distal end of the shuttle.

11. A stent delivery device for delivering a plurality of stent segments to a treatment site, the device comprising:
a catheter shaft having a proximal end and a distal end;
an expandable member coupled with the catheter shaft near the distal end;
an axially movable sheath disposed over at least part of the catheter shaft and the expandable member;
a shuttle disposed over at least part of the catheter shaft and the expandable member, at least part of the shuttle being radially expandable;
a plurality of stent segments slidably disposed along the shuttle; and
a stent pushing member disposed over the shuttle, proximal to the plurality of stent segments, for advancing the stent segments distally along the shuttle;
wherein moving the sheath axially toward the proximal end of the catheter shaft exposes at least part of the expandable member, allowing it to expand against the shuttle to cause the shuttle to radially expand, causing at least one of the plurality of stent segments to expand.

12. A device as in claim 11, wherein the shuttle is slidably disposed over at least part of the catheter shaft and the expandable member.

13. A device as in claim 11, wherein the shuttle is fixedly disposed over at least part of the catheter shaft and the expandable member.

14. A device as in claim 11, wherein the shuttle is disposed over the sheath.

15. A device as in claim 11, wherein the sheath is disposed over the shuttle.

16. A device as in claim 11, wherein the sheath is adapted to expose a first portion of the expandable member to deploy a first selected number of stent segments.

17. A device as in claim 16, wherein the sheath is adapted to further expose at least a second portion of the expandable member to deploy a second selected number of stent segments.

18. A device as in claim 11, wherein the shuttle further comprises an abutment near a distal end of the shuttle for preventing the plurality of stent segments from being advanced beyond the distal end of the shuttle.

19. A device as in claim 11, further including at least one valve member coupled with the sheath for selectively retaining at least one stent segment within the sheath.

20. A device as in claim 11, wherein the stent pushing member is configured to engage a proximal stent segment disposed at a proximal end of the plurality of stent segments.

21. A method for delivering a plurality of stent segments to a treatment site, the method comprising:
positioning a distal portion of a stent delivery catheter device at the treatment site; and
moving a sheath of the catheter device proximally to expose at least part of an expandable member on the catheter device, thus allowing the exposed expandable member to expand against an expandable shuttle of the catheter device to deploy at least one of the plurality of stent segments.

22. A method as in claim 21, wherein deploying the at least one stent segment comprises deploying a first plurality of stent segments.

23. A method as in claim 22, wherein deploying the first plurality of stent segments comprises:
selecting a number of stent segments desired to be deployed; and
moving the sheath to a position along the catheter device to deploy the selected number of stent segments.

24. A method as in claim 22, further comprising moving the sheath farther proximally to further expose the expandable member to allow it to expand against the expandable shuttle to deploy a second plurality of stent segments.

25. A method as in claim 24, further comprising moving the sheath farther proximally to further expose the expandable member to allow it to expand against the expandable shuttle to deploy a third plurality of stent segments.

26. A method as in claim 21, further comprising moving the plurality of stent segments in a distal direction along the shuttle, using a stent pushing member disposed over the shuttle proximal to the stent segments.

27. A method for delivering a plurality of stents to a treatment site, the method comprising:
positioning a distal portion of a stent delivery catheter device at the treatment site; and
moving a sheath of the catheter device proximally to expose at least part of an expandable member on the catheter device, thus allowing the exposed expandable member to expand against an expandable shuttle of the catheter device to deploy at least a first stent of the plurality of stents.

28. A method as in claim 27, wherein the first stent comprises a selected number of stent segments.

29. A method as in claim 27, further comprising moving the sheath farther proximally to further expose the expandable member to allow it to expand against the expandable shuttle to deploy a second stent.

30. A method as in claim 29, wherein the first and second stents have different lengths.

31. A method as in claim 29, wherein the first and second stents have different shapes.

32. A method as in claim 29, wherein the first stent comprises a first selected number of stent segments and the second stent comprises a second selected number of stent segments.

33. A method as in claim 29, further comprising moving the sheath farther proximally to further expose the expandable member to allow it to expand against the expandable shuttle to deploy a third stent.

34. A method as in claim 33, further comprising moving the plurality of stent segments in a distal direction along the shuttle, using a stent pushing member disposed over the shuttle proximal to the stent segments.
